(19) United States
(12) Patent Application Publication
(10) Pub. No.: US 2014/0234365 A1
(43) Pub. Date: Aug. 21, 2014

(54) 2-HYDROXYHIPPURIC ACID ANALOGS, AND METHODS FOR THEIR SYNTHESIS AND USE

(71) Applicant: ALERE SAN DIEGO, INC., San Diego, CA (US)

(72) Inventor: Mariusz Banaszczyk, San Marcos, CA (US)

(73) Assignee: ALERE SAN DIEGO, INC., San Diego, CA (US)

(21) Appl. No.: 14/347,160

(22) PCT Filed: Sep. 27, 2012
(86) PCT No.: PCT/US12/57471

§ 371 Date: Mar. 25, 2014

Related U.S. Application Data
(60) Provisional application No. 61/540,940, filed on Sep. 29, 2011.

(51) Int. Cl. A61K 47/48; C07D 333/32; G01N 33/53; C07C 235/50

(52) U.S. Cl. CPC A61K 47/48284; C07C 235/50; C07D 333/32; A61K 47/4833; G01N 33/5308
USPC 424/193.1; 564/153; 530/350; 536/23.1; 549/63; 530/400; 530/363; 435/188; 436/501; 435/7.93

(57) ABSTRACT

The present invention relates to novel 2-hydroxyhippuric acid analogs, and methods for their synthesis and use. Such analogs are designed to provide a protected or functional moiety such as a free thiol (—SH) group or a protected thiol group, thereby providing a convenient linkage chemistry for coupling under mild conditions to a suitable group on a target protein, polypeptide, solid phase or detectable label.

### CROSS-REFERENCE TO RELATED APPLICATIONS

The present invention claims priority of U.S. Provisional Application No. 61/540,940 filed Sep. 29, 2011, which is hereby incorporated by reference in its entirety including all tables, figures, and claims.

### FIELD OF THE INVENTION

The present invention relates to novel 2-hydroxyhippuric acid analogs useful for preparing conjugates comprising, inter alia, proteins, polypeptides, and labels; to conjugates comprising such 2-hydroxyhippuric acid analogs, and to methods for their synthesis and use.

### BACKGROUND OF THE INVENTION

The following discussion of the background of the invention is merely provided to aid the reader in understanding the invention and is not admitted to describe or constitute prior art to the present invention.

Aspirin (acetylsalicylic acid) is part of a group of medications referred to as nonsteroidal anti-inflammatory drugs (NSAIDs). Aspirin is rapidly metabolized in the body to salicylic acid, and is then further metabolized to a variety of compounds, including 2-hydroxyhippuric acid and a variety of glucuronide conjugates. 2-hydroxyhippuric acid (also known as salicyluric acid):

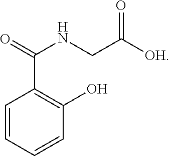 [SMILES: O=C(O)CNC(=O)c1ccccc1O]

2-hydroxyhippuric acid is a glycine conjugate of salicylic acid and represents the major urinary excretion product of aspirin.

It has been reported that appendicitis may be detected in human beings suspected of having appendicitis by determining a threshold level of 10 mg/liter of 2-hydroxyhippuric acid in the urine of such humans. This threshold level has been determined by qualitative, semiquantitative, or quantitative methods including HPLC (high pressure liquid chromatography), TLC (thin layer chromatography), radioimmunoassay, colorimetric tests, NMR (nuclear magnetic resonance), mass spectrometry, electrophoresis, and enzymatic tests.

In developing a binding assay for 2-hydroxyhippuric acid, the artisan must consider that samples may contain salicylic acid and other metabolites of aspirin. Thus, immunogenic and label conjugates should be designed to present 2-hydroxyhippuric acid in a manner that permits specific recognition and discrimination of molecules which differ only slightly in structure. Analogs for use in preparing such conjugates should also be designed to provide convenient attachment to various proteins, polypeptides, and labels under mild conditions.

### BRIEF SUMMARY OF THE INVENTION

It is an object of the invention to provide novel 2-hydroxyhippuric acid analogs, and methods for their synthesis and use. Such analogs are designed to provide a reactive thiol (—SH) group, providing a linkage chemistry for convenient coupling to a suitable group on a target protein, polypeptide, or label.

In a first aspect then, the invention relates to compounds (or salts thereof) having the following general formula:

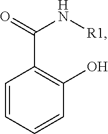

where R1 is a linkage chemistry which provides a functional moiety selected from the group consisting of protected or unprotected sulfhydryl moieties, protected or unprotected amine moieties, primary amine-reactive moieties, sulfhydryl-reactive moieties, photoreactive moieties, carboxyl-reactive moieties, arginine-reactive moieties, and carbonyl-reactive moieties.

In certain preferred embodiments, R1 is a linking group having the structure

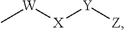

where
W is C₀₋₄ unsubstituted alkyl;
X is an optionally present C(O);
Y is an optionally substituted C₀₋₄ alkyl or N(H)—C₀₋₆ alkyl, and is optionally present; and
Z is a functional moiety selected from the group consisting of protected or unprotected sulfhydryl moieties, protected or unprotected amine moieties, primary amine-reactive moieties, sulfhydryl-reactive moieties, photoreactive moieties, carboxyl-reactive moieties, arginine-reactive moieties, and carbonyl-reactive moieties.

A variety of linkage chemistries have been described for the attachment of a particular molecule of interest, often for purposes developing binding assay (e.g., immunoassay) reagents. Thus, molecules may be coupled via a selected linkage chemistry for solid-phase immobilization, conjugation of haptens to immunogenic carrier molecules, preparation of antibody-detectable label conjugates, immunotoxins and other labeled protein and nucleic acid reagents, etc. Such linkage chemistries often provide the molecule of interest with one or more functional groups that couple to amino acid side chains of peptides. Among other characteristics, these “linkage reagents” may be classified on the basis of the following:

1. Functional group(s) and chemical specificity;

2. length and composition of the cross-bridge;

3. whether the functional group(s) react chemically or photochemically; and

4. whether the resultant linkage is cleavable.

Reactive groups that can be targeted using linkage chemistries include primary amines, sulfhydryls, carbonyls, carbohydrates and carboxylic acids. In addition, many reactive groups can be coupled nonselectively using a cross-linker such as photoreactive phenyl azides.

The choice of functional moiety may be varied by the artisan, depending on the desired length and composition for a crossbridge to a protein, polypeptide or label, and whether the reactive group is in free or in protected form. In the latter case, a wide variety of thiol protective groups are known in the art. See, e.g., standard reference works such as Greene and Wuts, PROTECTIVE GROUPS IN ORGANIC SYNTHESIS, 3^{rd} edition, John Wiley & Sons Inc., 1999, which is hereby incorporated by reference in its entirety. As described herein, Z preferably provides a thiol or a protected thiol at its terminus. Suitable thiol protective groups include dithiocarbamets, thioesters, thioethers, thiolactones, symmetrical and unsymmetrical disulfides, and sulfenyls.

In preferred embodiments, the functional moiety is a 5- or 6-member cyclic thiolactone, an optionally substituted C₁₋₄ alkyl thiol, or an optionally substituted thioester having the structure

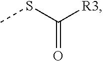

where R3 is selected from the group consisting of optionally substituted C₁₋₄ alkyl, optionally substituted C₁₋₄ alkoxy, and optionally substituted aryl.

In each of the embodiments described herein, substitution(s), when present, may be independently selected from the group consisting of C₁₋₆ alkyl straight or branched chain, benzyl, halogen, trihalomethyl, C₁₋₆ alkoxy, —NO₂, —NH₂, —OH, ═O, —COOR′ where R′ is H or lower alkyl, —CH₂OH, and —CONH₂.

In those embodiments where R1 is a linking group providing an unprotected thiol, X is most preferably C₀₋₄ unsubstituted alkyl, Y is most preferably absent or N(H), and Z is most preferably a C₁₋₄ alkyl thiol. In particularly preferred embodiments, Z preferably has the structure

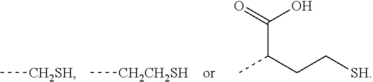

In a related aspect, the present invention relates to dimerized forms of the foregoing compounds, wherein the dimers are formed by disulfide bonding of the thiol or protected thiol in two compounds of the invention.

Most preferred compounds from the various embodiments described above may be one or more of the following:

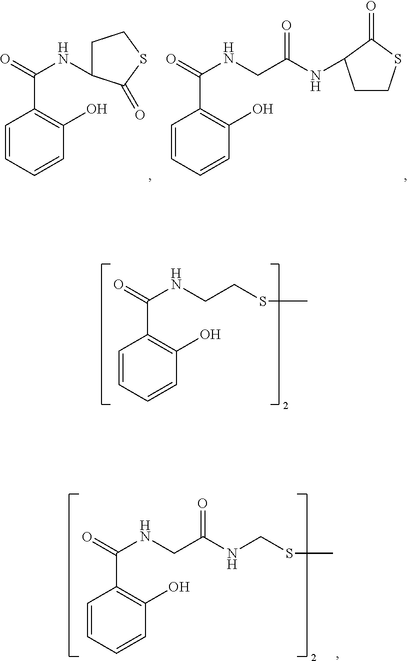

or salts thereof.

In a related aspect, the invention relates to compositions comprising one or more of the foregoing compounds (or their salts) covalently linked through the terminal thiol provided by R1 and/or R2 to a protein, polypeptide, label, or other molecule, referred to herein as “2-hydroxyhippuric acid analog conjugates.” These conjugates have the following general formula:

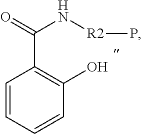

where
R2 is a linkage chemistry and P is a protein, polypeptide, label, or other molecule, wherein R2 and P are covalently linked. In exemplary embodiments, this covalent linkage is provided by a disulphide bond formed between a thiol on R2 and a thiol present on P.

In certain preferred embodiments, R2 is a linking group having the structure

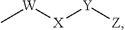

where
W is C₀₋₄ unsubstituted alkyl;
X is an optionally present C(O);
Y is an optionally substituted C₀₋₄ alkyl or N(H)—C₀₋₆ alkyl, and is optionally present; and
Z is a moiety providing a thiol which is covalently bound to a thiol present on P.

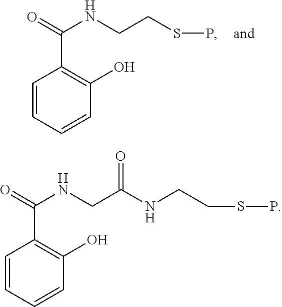

In the case of compounds of the invention comprising an unprotected thiol, the compounds may be directly linked to an appropriate target protein, polypeptide, label, or other molecule to form a conjugate via a thiol naturally occurring in the target molecule, or by adding a thiol through the addition of any thiol-directed coupling group on the target molecule. Exemplary thiol-directed coupling groups are described hereinafter, and methods for incorporating such coupling groups into target molecules for conjugation to the compounds described above are well known in the art. In the case of compounds of the invention comprising a protected thiol, removal of the protective group provides a free thiol, which is then linked to any thiol-directed coupling group on the target molecule in a similar fashion.

Preferred coupling groups on target molecules are maleimides, which are linked according to the following reaction scheme:

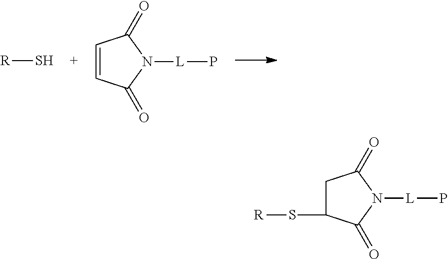

where R—SH is a compound of the invention comprising a free thiol (either as a free thiol or following deprotection of a protected thiol), L is a linkage chemistry, and P is a target protein, polypeptide, label, or other molecule. L is preferably C₁₋₁₀ alkylene straight or branched chain comprising from 0-4 backbone (i.e., non-substituent) heteroatoms, optionally substituted with from 1 to 4 substituents independently selected from the group consisting of C₁₋₆ alkyl straight or branched chain, —NO₂, —NH₂, ═O, halogen, trihalomethyl, C₁₋₆ alkoxy, —OH, —CH₂OH, and —C(O)NH₂.

In certain embodiments, P is a protein, most preferably an antigenic protein which can be used to raise an immune response to an epitope on the compound of the invention using a so-called “hapten-carrier” immunogen. Common carrier proteins include bovine serum albumin, keyhole limpet hemocyanin, ovalbumin, etc. Protocols for conjugation of haptens to carrier proteins may be found in ANTIBODIES: A LABORATORY MANUAL, E. Harlow and D. Lane, eds., Cold Spring Harbor Laboratory (Cold Spring Harbor, N.Y., 1988) pp. 78-87, which is hereby incorporated by reference.

Alternatively, P may preferably be a detectable label. Preferred detectable labels may include molecules or larger structures that are themselves detectable (e.g., fluorescent moieties, electrochemical labels, metal chelates, latex particles, etc.), as well as molecules that may be indirectly detected by production of a detectable reaction product (e.g., enzymes such as horseradish peroxidase, alkaline phosphatase, etc.) or by a specific binding molecule which itself may be detectable (e.g., biotin, avidin, streptavidin, digoxigenin, maltose, oligohistidine, 2,4-dintrobenzene, phenylarsenate, ssDNA, dsDNA, etc.). Exemplary conjugation to such detectable labels is described hereinafter. Particularly preferred detectable labels are fluorescent latex particles.

The foregoing lists of suitable target molecules are not meant to be limiting. Further exemplary embodiments are described hereinafter. In addition, numerous other classes of suitable targets, including peptide hormones, therapeutic proteins, antibodies, antibody fragments, single-chain variable region fragments, small molecules, nucleic acids, oligosaccharides, polysaccharides, cyclic polypeptides, peptidomimetics, aptamers and solid phases are known in the art.

While a conjugation target may be conjugated 1:1 with an 2-hydroxyhippuric acid analog of the invention, an individual target may also comprise more than 1 conjugation site, and hence more than 1 compound of the invention conjugated thereto. In preferred embodiments, a conjugation target comprises at least 102-hydroxyhippuric acid analog moieties covalently bound thereto, more preferably at least 30, still more preferably at least 50, and most preferably at least 100.

In still other related aspects, the present invention relates to methods for the production and use of the 2-hydroxyhippuric acid analog conjugates of the present invention.

Such methods can comprise contacting one or more compounds of the invention comprising a free thiol with one or more target molecules comprising one or more thiol-directed coupling groups, under conditions where the free thiol(s) react with the thiol-directed coupling group(s) to form one or more conjugates. Conditions for such reactions are dependent upon the thiol-directed coupling group(s) selected, and are well known to the skilled artisan. Exemplary conditions are described hereinafter.

Such methods may further comprise the step of deprotecting a protected thiol from one or more compounds of the invention prior to said contacting step, and/or attaching one or more thiol-directed coupling groups to a protein, polypeptide, label, or other molecule to form an appropriate conjugation target. In the latter case, this may comprise the use of bifunctional cross-linkers that provide an appropriate thiol-directed coupling group at one site in the molecule, and a second coupling group for attachment to the protein, polypeptide, label, or other molecule of interest. Numerous bifunctional cross-linkers are well known to those of skill in the art.

Regarding the use of such 2-hydroxyhippuric acid analog conjugates, the present invention relates to methods for preparing an antibody. These methods comprise using one or more conjugates as an immunogen to stimulate an immune response. As described above, an antibody may be raised against an immunogen having the structure

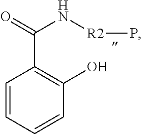

wherein R2 is a linkage chemistry and P is a carrier polypeptide, wherein R2 and P are covalently linked, preferably by a disulphide bond formed between a thiol on R2 and a thiol present on P.

In certain embodiments, methods may comprise administering one or more conjugates of the invention in a suitable immunization protocol, and separating an appropriate antibody from a body fluid of the animal. Exemplary protocols for preparing immunogens, immunization of animals, and collection of antiserum may be found in ANTIBODIES: A LABORATORY MANUAL, E. Harlow and D. Lane, eds., Cold Spring Harbor Laboratory (Cold Spring Harbor, N.Y., 1988) pp. 55-120, which is hereby incorporated by reference. Alternatively, the 2-hydroxyhippuric acid analog conjugates of the present invention may be used in phage display methods to select phage displaying on their surface an appropriate antibody, followed by separation of nucleic acid sequences encoding at least a variable domain region of an appropriate antibody. Phage display methods are well known to those of skill in the art. Such methods may use immunized or unimmunized animals as a source of nucleic acids to form the phage display library. Antibodies prepared in this manner may preferably find use as therapeutic molecules and/or as receptors in receptor binding assays.

Preferably, such antibodies bind 2-hydroxyhippuric acid with an affinity that is at least a factor of 5, more preferably at least a factor of 10, still more preferably at least a factor of 30, and most preferably at least a factor of 50 or more, than an affinity for salicylic acid and/or gentisic acid.

Antibodies prepared in this manner may be used as specific binding reagents in immunoassays for determining 2-hydroxyhippuric acid concentrations in samples. By way of example, a method can comprise performing a competitive immunoassay in which a conjugate having the structure

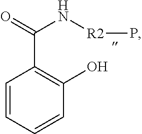

wherein R2 is a linkage chemistry and P is a detectable label, wherein R2 and P are covalently linked, preferably by a disulphide bond formed between a thiol on R2 and a thiol present on P, competes with 2-hydroxyhippuric acid in said sample for binding to an antibody, wherein a signal obtained from said assay is indicative of the concentration of 2-hydroxyhippuric acid in said sample; and
determining the concentration of 2-hydroxyhippuric acid in said sample from the assay signal. Preferably, immunoassays provide a signal that is at least a factor of 5, more preferably at least a factor of 10, still more preferably at least a factor of 30, and most preferably at least a factor of 50 or more for 10 μg/mL 2-hydroxyhippuric acid, compared to the signal obtained from 10 μg/mL, and more preferably 1000 μg/mL, salicylic acid and/or gentisic acid.

As described above, such assays can find use in methods for diagnosing appendicitis in a subject. These methods comprise performing a competitive immunoassay as described herein; and correlating the concentration of 2-hydroxyhippuric acid determined to the presence or absence of appendicitis in the subject.

Other embodiments of the invention will be apparent from the following detailed description, exemplary embodiments, and claims.

### DETAILED DESCRIPTION OF THE INVENTION

The present invention relates in part to amino acid analogs and methods for their production and use, particularly for preparing cross-linkable thiol-containing 2-hydroxyhippuric acid analogs for conjugation to another molecule, and for use of such conjugates for preparing reagents for immunoassays that detect 2-hydroxyhippuric acid. The analogs of the present invention are particularly well suited for producing antibodies and labels for use in receptor binding assays for 2-hydroxyhippuric acid that can distinguish 2-hydroxyhippuric acid from salicylic acid.

For the sake of clarity, definitions for the following terms regarding the compounds of the present invention are provided.

As used herein, the term “aryl” refers to an optionally substituted aromatic group with at least one ring having a conjugated pi-electron system, containing up to two conjugated or fused ring systems. Aryl includes carboxylic aryl, heterocyclic aryl and biaryl groups, all of which may be optionally substituted. Preferably, the aryl is either optionally substituted phenyl, optionally substituted pyridyl, optionally substituted benzothiopyranyl, optionally substituted carbazole, optionally substituted naphthyl, optionally substituted tetrahydronaphthyl. While “aryl” is most preferably a monocyclic carbocyclic aromatic ring having 5 or 6 ring atoms (and is most preferably phenyl), the aryl or heteroaryl Ar group (formed into an arylene or heteroarylene in the crosslinkers described herein by elaboration from a ring atom) generally may contain up to ten ring atoms, although the skilled artisan will recognize that aryl groups with more than ten ring atoms are within the scope of the invention. The ring systems encompassed by Ar can contain up to four heteroatoms, independently selected from the group consisting of N, S, and O.

Monocyclic aryl groups include, but are not limited to: phenyl, thiazoyl, furyl, pyranyl, 2H-pyrrolyl, thienyl, pyrroyl, imidazoyl, pyrazoyl, pyridyl, pyrazinyl, pyrimidinyl, and pyridazinyl moieties. Fused bicyclic Ar groups include, but are not limited to: benzothiazole, benzimidazole, 3H-indolyl, indolyl, indazoyl, purinyl, quinolizinyl, isoquinolyl, quinolyl, phthalizinyl, naphthyridinyl, quinazolinyl, cinnolinyl, isothiazolyl, quinoxalinyl indolizinyl, isoindolyl, benzothienyl, benzofuranyl, isobenzofuranyl, and chromenyl moieties.

As used herein, the term “heteroatom” refers to non-carbon, non-hydrogen atoms such as N, O, and S.

The aryl group may also be optionally substituted by replacement of one or more hydrogen atoms by another chemical moiety. Preferred substituents include C₁₋₆ alkyl straight or branched (e.g. isopropyl) chain, halogen, trihalomethyl, alkoxy, NO₂, NH₂, OH, —COOR′, where R′ is H or lower alkyl, CH₂OH, and CONH₂.

As used herein, the term “alkyl” refers to a saturated aliphatic hydrocarbon including straight chain and branched chain groups. Preferably, the alkyl group has 1 to 20 carbon atoms. More preferably, it is a medium alkyl (having 1 to 10 carbon atoms). Most preferably, it is a lower alkyl (having 1 to 4 carbon atoms). The alkyl group may be substituted or unsubstituted.

As used herein, the term “alkoxy” group refers to both an —O-alkyl and an —O-cycloalkyl group; preferably an alkoxy group refers to a lower alkoxy, and most preferably methoxy or ethoxy.

As used herein, the term “thiolactone” refers to a cyclic hydrocarbon having 5 or 6 ring atoms, one of which is an S heteroatom, and where the heteroatom is adjacent to a carbon substituted with a ═O.

As used herein, the term “thioester” refers to an organic compound having the structure R—S—C(O)—R′.

As used herein, the term “alkyl thiol” refers to an alkyl group containing an —SH group. Thiols are also referred to as “thio alcohols” and “sulfhydryls.”

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The term “polypeptide” as used herein refers to a molecule having a sequence of amino acids linked by peptide bonds. This term includes proteins, fusion proteins, oligopeptides, cyclic peptides, and polypeptide derivatives. Antibodies and antibody derivatives are discussed above in a separate section, but antibodies and antibody derivatives are, for purposes of the invention, treated as a subclass of the polypeptides and derivatives. The term protein refers to a polypeptide that is isolated from a natural source, or produced from an isolated cDNA using recombinant DNA technology, and that has a sequence of amino acids having a length of at least about 200 amino acids.

The term “nucleic acids” as used herein shall be generic to polydeoxyribonucleotides (containing 2′-deoxy-D-ribose or modified forms thereof), to polyribonucleotides (containing D-ribose or modified forms thereof), and to any other type of polynucleotide which is an N-glycoside of purine or pyrimidine bases, or modified purine or pyrimidine bases.

The term “aptamer” as used herein is a single-stranded or double-stranded oligodeoxyribonucleotide, oligoribonucleotide or modified derivatives that specifically bind and alter the biological function of a target molecule. The target molecule is defined as a protein, peptide and derivatives thereof. The aptamer is capable of binding the target molecule under physiological conditions. An aptamer effect is distinguished from an antisense effect in that the aptameric effects are induced by binding to the protein, peptide and derivative thereof and are not induced by interaction or binding under physiological conditions with nucleic acid.

The term “polysaccharide” as used herein refers to a molecule comprising more than 10 glycosidically linked monosaccharide residues, while the term “oligosaccharide” refers to a molecule comprising from 2-10 glycosidically linked monosaccharide residues.

The term “small molecule” includes any molecule having a molecular weight less than about 5,000 daltons (Da), preferably less than about 2,500 Da, more preferably less than 1,000 Da, most preferably less than about 500 Da.

Functional Moieties

Chemical cross-linkers are valuable tools for preparing antibody-detectable label conjugates, immunotoxins and other labeled protein and nucleic acid reagents. These reagents may be classified on the basis of the following:

1. Functional groups and chemical specificity;

2. length and composition of the cross-bridge;

3. whether the cross-linking groups are similar (homobifunctional) or different (heterobifunctional);

4. whether the groups react chemically or photochemically;

5. whether the reagent is cleavable; and

6. whether the reagent can be radiolabeled or tagged with another label.

7. whether the reagent can incorporate PEG cross-bridge or other polymer.

As the exemplary compounds of the present invention provide an available thiol to act as an attachment point, targets may be prepared to provide an appropriate thiol-reactive site. Cross-linking reagents that couple through sulfhydryls (thiols) are available from many commercial sources. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Such reagents may be bifunctional, in that a second site on the reagent is available for use in modifying a conjugation target to incorporate the thiol-reactive site. In addition to thiols, reactive groups that can be targeted using a cross-linker include primary amines, carbonyls, carbohydrates and carboxylic acids. In addition, many reactive groups can be coupled nonselectively using a cross-linker such as photoreactive phenyl azides. Thus, a two-step strategy allows for the coupling of a protein that can tolerate the modification of its amines to an 2-hydroxyhippuric acid analog of the invention. For suitable reagents, see Pierce 2003-2004 Applications Handbook and Catalog #1600926, which is hereby incorporated by reference. Cross-linkers that are amine-reactive at one end and sulfhydryl-reactive at the other end are quite common. If using heterobifunctional reagents, the most labile group is typically reacted first to ensure effective cross-linking and avoid unwanted polymerization.

Many factors must be considered to determine optimum cross-linker-to-target molar ratios. Depending on the application, the degree of conjugation is an important factor. For example, when preparing immunogen conjugates, a high degree of conjugation is normally desired to increase the immunogenicity of the antigen. However, when conjugating to an antibody or an enzyme, a low-to-moderate degree of conjugation may be optimal to ensure that the biological activity of the protein is retained. It is also important to consider the number of reactive groups on the surface of the protein. If there are numerous target groups, a lower cross-linker-to-protein ratio can be used. For a limited number of potential targets, a higher cross-linker-to-protein ratio may be required. This translates into more cross-linker per gram for a small molecular weight protein.

Conformational changes of proteins associated with a particular interaction may also be analyzed by performing cross-linking studies before and after the interaction. A comparison is made by using different arm-length cross-linkers and analyzing the success of conjugation. The use of cross-linkers with different reactive groups and/or spacer arms may be desirable when the conformation of the protein changes such that hindered amino acids become available for cross-linking.

Cross-linkers are available with varying lengths of spacer arms or bridges connecting the reactive ends. The most apparent attribute of the bridge is its ability to deal with steric considerations of the moieties to be linked. Because steric effects dictate the distance between potential reaction sites for cross-linking, different lengths of bridges may be considered for the interaction. Shorter spacer arms are often used in intramolecular cross-linking studies, while intermolecular cross-linking is favored with a cross-linker containing a longer spacer arm.

The inclusion of polymer portions (e.g., polyethylene glycol (“PEG”) homopolymers, polypropylene glycol homopolymers, other alkyl-polyethylene oxides, bis-polyethylene oxides and co-polymers or block co-polymers of poly(alkylene oxides)) in cross-linkers can, under certain circumstances be advantageous. See, e.g., U.S. Pat. Nos. 5,643,575, 5,672,662, 5,705,153, 5,730,990, 5,902,588, and 5,932,462; and Topchieva et al., Bioconjug. Chem. 6: 380-8, 1995). For example, U.S. Pat. No. 5,672,662 discloses bifunctional cross-linkers comprising a PEG polymer portion and a single ester linkage. Such molecules are said to provide a half-life of about 10 to 25 minutes in water.

Designing a cross-linker involves selection of the functional moieties to be employed. The choice of functional moieties is entirely dependent upon the target sites available on the species to be crosslinked. Some species (e.g., proteins) may present a number of available sites for targeting (e.g., lysine s-amino groups, cysteine sulfhydryl groups, glutamic acid carboxyl groups, etc.), and selection of a particular functional moiety may be made empirically in order to best preserve a biological property of interest (e.g., binding affinity of an antibody, catalytic activity of an enzyme, etc.)

1. Coupling Through Amine Groups

Imidoester and N-hydroxysuccinimidyl (“NHS”) esters are typically employed as amine-specific functional moieties. NHS esters yield stable products upon reaction with primary or secondary amines. Coupling is efficient at physiological pH, and NHS-ester cross-linkers are more stable in solution than their imidate counterparts. Homobifunctional NHS-ester conjugations are commonly used to cross-link amine-containing proteins in either one-step or two-step reactions. Primary amines are the principle targets for NHS-esters. Accessible α-amine groups present on the N-termini of proteins react with NHS-esters to form amides. However, because α-amines on a protein are not always available, the reaction with side chains of amino acids become important. While five amino acids have nitrogen in their side chains, only the ε-amino group of lysine reacts significantly with NHS-esters. A covalent amide bond is formed when the NHS-ester cross-linking agent reacts with primary amines, releasing N-hydroxysuccinimide.

2. Coupling Through Sulfhydryl Groups

Maleimides, alkyl and aryl halides, α-haloacyls, and pyridyl disulfides are typically employed as sulfhydryl-specific functional moieties. The maleimide group is specific for sulfhydryl groups when the pH of the reaction mixture is kept between pH 6.5 and 7.5. At pH 7, the reaction of the maleimides with sulfhydryls is 1000-fold faster than with amines. Maleimides do not react with tyrosines, histidines or methionines. When free sulfhydryls are not present in sufficient quantities, they can often be generated by reduction of available disulfide bonds.

3. Coupling Through Carboxyl Groups

Carbodiimides couple carboxyls to primary amines or hydrazides, resulting in formation of amide or hydrazone bonds. Carbodiimides are unlike other conjugation reactions in that no cross-bridge is formed between the carbodiimide and the molecules being coupled; rather, a peptide bond is formed between an available carboxyl group and an available amine group. Carboxy termini of proteins can be targeted, as well as glutamic and aspartic acid side chains. In the presence of excess cross-linker, polymerization may occur because proteins contain both carboxyls and amines. No cross-bridge is formed, and the amide bond is the same as a peptide bond, so reversal of the cross-linking is impossible without destruction of the protein.

4. Nonselective Labeling

A photoaffinity reagent is a compound that is chemically inert but becomes reactive when exposed to ultraviolet or visible light. Arylazides are photoaffinity reagents that are photolyzed at wavelengths between 250-460 nm, forming a reactive aryl nitrene. The aryl nitrene reacts nonselectively to form a covalent bond. Reducing agents must be used with caution because they can reduce the azido group.

5. Carbonyl Specific Cross-Linkers

Carbonyls (aldehydes and ketones) react with amines and hydrazides at pH 5-7. The reaction with hydrazides is faster than with amines, making this useful for site-specific cross-linking. Carbonyls do not readily exist in proteins; however, mild oxidation of sugar moieties using sodium metaperiodate will convert vicinal hydroxyls to aldehydes or ketones.

Exemplary Applications for Use of Cross-Linkable 2-hydroxyhippuric Acid Analogs

1. Carrier Protein-Hapten/Peptide/Polypeptide Conjugates for Use as Immunogens

Numerous companies offer commercially available products in this area of immunological research. There are many cross-linkers used for the production of these conjugates, and the best choice is dependent on the reactive groups present on the hapten and the ability of the hapten-carrier conjugate to function successfully as an immunogen after its injection. Carbodiimides are good choices for producing peptide carrier conjugates because both proteins and peptides usually contain several carboxyls and primary amines. Other cross-linkers can also be used to make immunogen conjugates.

Adjuvants are mixtures of natural or synthetic compounds that, when administered with antigens, enhance the immune response. Adjuvants are used to (1) stimulate an immune response to an antigen that is not inherently immunogenic, (2) increase the intensity of the immune response, (3) preferentially stimulate either a cellular or a humoral response (i.e., protection from disease versus antibody production). Adjuvants have four main modes of action: enhanced antigen uptake and localization, extended antigen release, macrophage activation, and T and B cell stimulation. The most commonly used adjuvants fall into six categories: mineral salts, oil emulsions, microbacterial products, saponins, synthetic products and cytokines. A more extensive discussion of adjuvants and their use in immunization protocols is given in IMMUNOLOGY METHODS MANUAL, vol. 2, I. Lefkovits, ed., Academic Press, San Diego, Calif., 1997, ch. 13, which is hereby incorporated in its entirety

Small molecules such as 2-hydroxyhippuric acid are not usually immunogenic, even when administered in the presence of adjuvant. In order to generate an immune response to these compounds, it is often necessary to attach them to a protein or other compound, termed a carrier, that is immunogenic. When attached to a carrier protein the small molecule immunogen is called a hapten. Haptens are also conjugated to carrier proteins for use in immunoassays. The carrier protein provides a means of attaching the hapten to a solid support such as a microtiter plate or nitrocellulose membrane. When attached to agarose they may be used for purification of the anti-hapten antibodies. They may also be used to create a multivalent antigen that will be able to form large antigen-antibody complexes. When choosing carrier proteins, remember that the animal will form antibodies to the carrier protein as well as to the attached hapten. It is therefore important to select a carrier protein for immunization that is unrelated to proteins that may be found in the assay sample. If haptens are being conjugated for both immunization and assay, the two carrier proteins should be as different as possible. This allows the antiserum to be used without having to isolate the anti-hapten antibodies from the anti-carrier antibodies.

Keyhole limpet hemocyanin (KLH) is a respiratory protein found in mollusks. Its large size makes it very immunogenic, and the large number of lysine residues available for conjugation make it very useful as a carrier for haptens such as 2-hydroxyhippuric acid. The phylogenic separation between mammals and mollusks increases the immunogenicity and reduces the risk of cross-reactivity between antibodies against the KLH carrier and naturally occurring proteins in mammalian samples.

2. Solid-Phase Immobilization

The 2-hydroxyhippuric acid analogs and/or conjugates of the present invention can be immobilized on solid-phase matrices for use as affinity supports or for sample analysis. Similarly, antibodies or their binding fragments made or selected using the 2-hydroxyhippuric acid analogs and/or conjugates of the present invention can also be immobilized on solid-phase matrices. The term “solid phase” as used herein refers to a wide variety of materials including solids, semi-solids, gels, films, membranes, meshes, felts, composites, particles, papers and the like typically used by those of skill in the art to sequester molecules. The solid phase can be non-porous or porous. Suitable solid phases include those developed and/or used as solid phases in solid phase binding assays. See, e.g., chapter 9 of Immunoassay, E. P. Dianiandis and T. K. Christopoulos eds., Academic Press: New York, 1996, hereby incorporated by reference. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. See, e.g., Leon et al., Bioorg. Med. Chem. Lett. 8: 2997, 1998; Kessler et al., Agnew. Chem. Int. Ed. 40: 165, 2001; Smith et al., J. Comb. Med. 1: 326, 1999; Orain et al., Tetrahedron Lett. 42: 515, 2001; Papanikos et al., J. Am. Chem. Soc. 123: 2176, 2001; Gottschling et al., Bioorg. Med. Chem. Lett. 11: 2997, 2001.

Surfaces such as those described above may be modified to provide linkage sites, for example by bromoacetylation, silation, addition of amino groups using nitric acid, and attachment of intermediary proteins, dendrimers and/or star polymers. This list is not meant to be limiting, and any method known to those of skill in the art may be employed.

3. Detectable Label Conjugates

Biological assays require methods for detection, and one of the most common methods for quantitation of results is to conjugate an enzyme, fluorophore or other detectable label to the molecule under study (e.g., using one or more 2-hydroxyhippuric acid analogs of the invention), which may be immobilized for detection by a receptor molecule that has affinity for the molecule. Alternatively, the receptor to the molecule under study (e.g., an antibody or binding fragment thereof made or selected using the analogs or conjugates of the invention) may be conjugated to an enzyme, fluorophore or other detectable label. Enzyme conjugates are among the most common conjugates used. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

Particularly preferred detectable labels are fluorescent latex particles such as those described in U.S. Pat. Nos. 5,763,189, 6,238,931, and 6,251,687; and International Publication WO95/08772, each of which is hereby incorporated by reference in its entirety. Exemplary conjugation to such particles is described hereinafter.

Use of 2-hydroxyhippuric Acid Analogs in Receptor Binding Assays

The 2-hydroxyhippuric acid analogs and conjugates of the present invention may be advantageously used in receptor binding assays. Receptor binding assays include any assay in which a signal is dependent upon specific binding of an analyte to a cognate receptor, and include immunoassays, ligand-receptor assays, and nucleic acid hybridization assays.

The presence or amount of an analyte is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

Numerous methods and devices are well known to the skilled artisan for the practice of receptor binding assays. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize detectably labeled molecules and antibody solid phases in various sandwich, competitive, or non-competitive assay formats, to generate a signal that is related to the presence or amount of an analyte of interest. One skilled in the art also recognizes that robotic instrumentation including but not limited to Beckman Access, Abbott AxSym, Roche ElecSys, Dade Behring Stratus systems are among the immunoassay analyzers that are capable of performing such immunoassays. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. As described herein, preferred assays utilize an antibody raised against an 2-hydroxyhippuric acid analog conjugate (wherein the antibody is coupled to a solid phase or a detectable label), and/or an 2-hydroxyhippuric acid analog conjugated to a detectable label, and/or an 2-hydroxyhippuric acid analog conjugated to a solid phase.

In its simplest form, an assay device according to the invention may comprise a solid surface comprising receptor(s) that specifically bind one or more analytes of interest (e.g., 2-hydroxyhippuric acid). For example, antibodies may be immobilized onto a variety of solid supports, such as magnetic or chromatographic matrix particles, the surface of an assay plate (such as microtiter wells), pieces of a solid substrate material or membrane (such as plastic, nylon, paper), and the like using the cross-linkers of the present invention. In similar fashion, an assay device may comprise a solid surface comprising one or more of the 2-hydroxyhippuric acid analogs described herein immobilized thereon.

The analysis of a plurality of analytes may be carried out separately or simultaneously with one test sample. For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses or protein chips perform simultaneous assays of a plurality of analytes on a single surface. Particularly useful physical formats comprise surfaces having a plurality of discrete, addressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

### EXAMPLES

### Example 1

### Synthesis of N,N′-(2,2′-(2,2′-disulfanediylbis(ethane-2,1-diyl)bis(azanediyl))bis(2-oxoethane-2,1-diyl))bis(2-hydroxybenzamide) (2-Hydroxyhippuric-Cystamine)

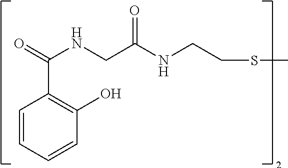

To a mixture of 2-hydroxyhippuric acid (1 g, 5.12 mmol), cystamine dihydrochloride (0.58 g, 2.56 mmol) and O-(7-Azabenzotriazol-1-yl)-N,N,N′,N′-tetramethyluronium hexafluorophosphate (HATU, 1.9 g, 5.12 mmol) in dry tetrahydrofuran (10 mL) was added diisopropylethylamine (DIEA, 1.79 mL, 10.25 mmol). The resulting mixture was heated at 60° C. for 1 hour. The reaction mixture was cooled down to room temperature and then concentrated to dryness in vacuo. The oil residue was extracted with ethyl acetate (100 mL) and 5% NaHCO3 (2×100 mL). The organic layer was separated and then washed with 1 N HCl (2×100 mL). The organic layer was separated and kept at room temperature overnight. The white solid was recrystallized from ethyl acetate to yield 0.84 g (65%). 1H NMR (DMSO-d6) δ 12.23 (2H, s), 9.07 (2H, t), 8.21 (2H, t), 7.87 (2H, d), 7.40 (2H, t), 6.90 (4H, m), 3.90 (4H, d), 3.37 (4H, q), 2.79 (4H, t).

### Example 2

### Synthesis of 2-hydroxy-N-(2-oxo-2-(2-oxotetrahydrothiophen-3-ylamino)ethyl)benzamide(2-Hydroxyhippuric-HCTL)

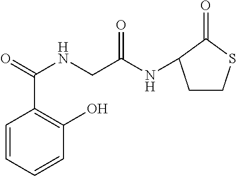 [SMILES: O=C(CNC(=O)c1ccccc1O)NC1CCSC1=O]

To a mixture of 2-hydroxyhippuric acid (1 g, 5.12 mmol), DL-homocysteine thiolactone hydrochloride (0.79 g, 5.12 mmol) and O-(7-Azabenzotriazol-1-yl)-N,N,N′, N′-tetramethyluronium hexafluorophosphate (HATU, 1.9 g, 5.12 mmol) in dry tetrahydrofuran (10 mL) was added diisopropylethylamine (DIEA, 1.79 mL, 10.25 mmol). The resulting mixture was heated at 60° C. for 1 hour. The reaction mixture was cooled down to room temperature and then concentrated to dryness in vacuo. The oil residue was extracted with ethyl acetate (100 mL) and 5% NaHCO3 (2×100 mL). The organic layer was separated and then washed with 1 N HCl (2×100 mL), brine (100 mL), dried over magnesium sulfate, filtered and evaporated under vacuum to yield 1.2 g (80%). 1H NMR (DMSO-d6) δ 12.25 (1H, s), 9.10 (1H, t), 8.40 (1H, d), 7.88 (1H, d), 7.41 (1H, t), 6.91 (2H, m), 4.65 (1H, m), 3.95 (2H, m), 3.39 (1H, m), 3.29 (1H, m), 2.43 (1H, m), 2.11 (1H, m).

### Example 3

### Synthesis of 2-hydroxy-N-(2-oxotetrahydrothiophen-3-yl)benzamide(Salicylic-HCTL)

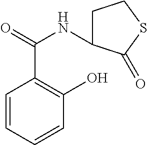 [SMILES: O=C(NC1CCSC1=O)c1ccccc1O]

A mixture of salicylic acid (1.38 g, 10 mmol) and 1-ethyl-3-(3-dimethylaminopropyl)carbodiimide (EDC, 1.95 mL, 11 mmol) was dissolved in acetonitrile (20 mL). To the mixture was added DL-homocysteine thiolactone hydrochloride (1.84 g, 12 mmol). The reaction mixture was then stirred at room temperature for overnight. The mixture was then concentrated to dryness in vacuo. The oil residue was extracted with ethyl acetate (3×100 mL) and phosphoric acid buffer pH=3 (100 mL). The combined organic layers were washed with brine (100 mL), dried over magnesium sulfate, filtered and evaporated under vacuum to yield 1.6 g (68%) as a gooey solid. A portion of product (100 mg) was purified by column chromatography on silica gel eluting with ethyl acetate/hexane (15:85, v/v) to give 24 mg as a white solid. 1H NMR (DMSO-d6) δ 12.25 (1H, s), 9.14 (1H, s), 7.84 (1H, d), 7.41 (1H, t), 6.90 (2H, m), 4.87 (1H, m), 3.46 (1H, m), 3.35 (1H, m), 2.53 (1H, m), 2.33 (1H, m).

### Example 4

### Synthesis of KLH-SMCC

Keyhole Limpet Hemocyanin (KLH, Calbiochem #374817, 50 mg/mL in glycerol) was passed through a 40 mL GH25 column equilibrated in 0.1M potassium phosphate, 0.1M borate, 0.15M sodium chloride buffer, pH 7.5 to remove glycerol. A 1.5-fold molar excess of N-ethylmaleimide was added, and the mixture incubated 30 minutes at room temperature. A 200-fold molar excess of sulfo-SMCC (Pierce #22322) from a 50 mM stock in distilled water was added while vortexing. Vortexing was continued for another 30 seconds, followed by incubation for 10 minutes at room temperature. A 100-fold molar excess of SMCC (Pierce #22360) from an 80 mM stock in acetonitrile was added while vortexing. 1M KOH was added to maintain a pH of between 7.2 and 7.4. The mixture was stirred at room temperature for 90 minutes. After 90 minutes incubation, KLH-SMCC was purified by gel filtration using a GH25 column equilibrated in 0.1M potassium phosphate, 0.02M borate, 0.15M sodium chloride buffer, pH 7.0.

### Example 5

### 2-hydroxyhippuric Derivative Conjugates

2-Hydroxyhippuric derivatives were conjugated to KLH-SMCC as follows. First, an S-acetyl-functionalized 2-Hydroxyhippuric derivative was deprotected by base hydrolysis to provide free thiol. The derivative (4-8 mg) was dissolved in 0.8 mL DMF-water solution (70:30 v/v) and 200 μL of 1 M KOH, and was incubated for 10 minutes at room temperature. The excess of the base was neutralized with a phosphate/hydrochloric acid buffer and pH brought to 7. Then, a 2-fold molar excess of 2-Hydroxyhippuric derivative (based on the concentration of SMCC in a particular batch of KLH-SMCC) was added to KLH-SMCC, and the mixture stirred for 90 minutes at room temperature. Conjugates were purified by exhaustive dialysis in PBS.

### Example 6

### Immunoassay for Detecting 2-hydroxyhippuric Acid

A competitive assay for detecting 2-hydroxyhippuric acid in urine was developed using microfluidic devices manufactured at Alere San Diego, Inc. essentially as described in WO98/43739, WO98/08606, WO98/21563, and WO93/24231. An anti-2-Hydroxyhippuric acid antibody was developed by phage display using a hapten-KLH conjugate as immunogen. This antibody was conjugated to a 0.13 μm maleimide-functionalized latex particle via a free cysteine residue on the antibody. The detection reagent consisted of a 0.50 μm fluorescence energy transfer latex particle (essentially as described in U.S. Pat. Nos. 5,763,189, 6,238,931, and 6,251,687; and International Publication WO95/08772) made according to Example 6. 130 mL (comprising 0.22% solids) of the antibody-particle conjugate was spotted onto the diagnostic lane of the microfluidic device, and 170 nL (comprising 0.4% solids) of the 2-Hydroxyhippuric acid-particle conjugate was applied to the device reaction chamber. Analytes were dissolved in deionized water, then diluted into pools of human plasma to achieve the desired final concentration. 210 μL of sample was applied to the device sample addition zone and allowed to run >15 minutes prior to reading the fluorescence in the TRIAGE (Biosite Incorporated) meter. A fluorescent signal was obtained by integrating the fluorescence as a function of distance from the device origin. The assay detected 2-Hydroxyhippuric acid at a concentration of 10 μg/mL, but does not appreciably detect the closely related compounds gentisic acid and salicylic acid at concentrations at least as high as 1 mg/mL.

The foregoing examples serve to illustrate the present invention. These examples are in no way intended to limit the scope of the invention.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

1. A compound or salt thereof, said compound having the structure: 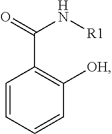
Wherein R1 is a linking group having the structure 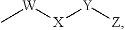
where
W is C₀₋₁₂ unsubstituted, substituted, liner or branched alkyl;
X is an optionally present C(O);
Y is an optionally substituted C₀₋₁₂ alkyl or N(H)—C₀₋₆ alkyl, and is optionally present; and
Z is a functional moiety selected from the group consisting of protected or unprotected sulfhydryl moieties, protected or unprotected amine moieties, primary amine-reactive moieties, sulfhydryl-reactive moieties, photoreactive moieties, carboxyl-reactive moieties, arginine-reactive moieties, and carbonyl-reactive moieties.

2. A compound or salt thereof, said compound having the structure: 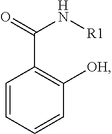
Wherein R1 is a linking group having the structure 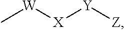
where
W is C₀₋₁₂ unsubstituted, substituted, liner or branched alkyl;
X is an optionally present C(O);
Y is an optionally substituted C₀₋₁₂ alkyl or N(H)—C₀₋₆ alkyl, and is optionally present; and
Z is a moiety providing a thiol or a protected thiol at its terminus.

3. A compound or salt thereof according to claim 2, wherein R1 is a thiolactone, disulfide or a thioester.

4. A compound or salt thereof according to claim 2, wherein R 1 is a thiolactone having 5 ring members.

5. A compound or salt thereof according to claim 2, wherein X is —C(O)—, Y is N(H)—C₀₋₆ alkyl and Z is a thiolactone.

6. A compound or salt thereof according to claim 2, wherein R1 has the structure —CH₂C(O)N(H)—Z.

7. A compound or salt thereof according to claim 2, wherein Z is an alkyl thiol.

8. A compound or salt thereof according to claim 2, selected from the group consisting of:

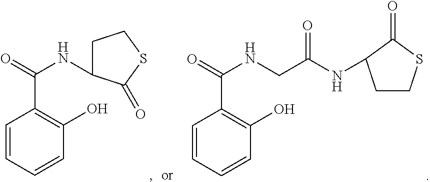 [SMILES: O=C(NC1CCSC1=O)c1ccccc1O ; O=C(CNC(=O)c1ccccc1O)NC1CCSC1=O]

9. A dimeric compound comprising two compounds according to claim 2, wherein the dimeric compound is formed by disulfide bonding of the thiol or protected thiol in two compounds.

10. A dimeric compound according to claim 9 selected from the group consisting of

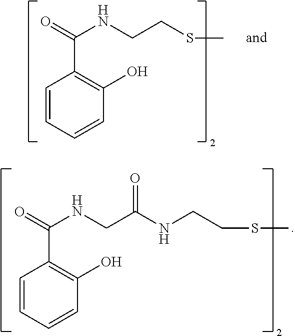

11. A conjugate comprising one or more compounds according to claim 1 covalently bound to a protein, polypeptide, detectable label, nucleic acid, or solid phase.

12. A conjugate according to claim 11, wherein the functional moiety is a sulfhydryl-reactive moiety selected from the group consisting of a maleimide, an alkyl halide, an aryl halide, an alpha-haloacyl, and a pyridyl disulfide.

13. A conjugate according to claim 12, wherein said sulfhydryl-reactive moiety is a maleimide, whereby said compound(s) are covalently bound to said protein, polypeptide, detectable label, nucleic acid, or solid phase to provide the following structure: 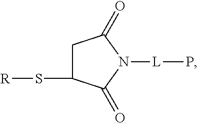
wherein R—S is a compound of the invention covalently bound through a thiol linkage, L is a linkage chemistry, and P is said protein, polypeptide, detectable label, nucleic acid, or solid phase, and wherein L is C₁₋₁₀ alkylene straight or branched chain comprising from 0-4 backbone heteroatoms, optionally substituted with from 1 to 4 substituents independently selected from the group consisting of C₁₋₆ alkyl straight or branched chain, —NO₂, —NH₂, ═O, halogen, trihalomethyl, C₁₋₆ alkoxy, —OH, —CH₂OH, and —C(O)NH₂.

14. A conjugate according to claim 12, wherein said detectable label is selected from the group consisting of an enzyme, a fluorophore, biotin, avidin, streptavidin, digoxigenin, maltose, oligohistidine, 2,4-dintrobenzene, phenylarsenate, and a fluorescent latex particle, nanogold particle.

15. A conjugate according to claim 12, wherein said protein is keyhole limpet hemocyanin or bovine serum albumin or BTG.

16. A conjugate according to claim 12, wherein said compound(s) are bound to a solid phase selected from the group consisting of a membrane, a cellulose-based paper, a polymeric particle, a latex particle, a paramagnetic particle, a gold particle, a glass substrate, a silicon substrate, a plastic substrate, and a multiple-well plate.

17. A method of preparing a conjugate, comprising:
contacting one or more compounds of claim 2 with a protein, polypeptide, detectable label, nucleic acid, or solid phase comprising one or more sulfhydryl-reactive moieties under conditions selected to provide covalent coupling of said compound(s) to said protein, polypeptide, detectable label, nucleic acid, or solid phase through a sulfhydryl group on the compound(s).

18. A method according to claim 17, wherein said compound(s) comprise a protected thiol, and said method further comprises deprotecting said thiol prior to or together with said contacting step.

19. A method according to claim 17, wherein said method further comprises introducing said one or more sulfhydryl-reactive moieties to said protein, polypeptide, detectable label, nucleic acid, or solid phase prior to said contacting step.

20. A method according to claim 19, wherein said one or more sulfhydryl-reactive moieties are selected from the group consisting of a maleimide, an alkyl halide, an aryl halide, an alpha-haloacyl, and a pyridyl disulfide, and said introducing step comprises coupling of said protein, polypeptide, detectable label, nucleic acid, or solid phase to one or more bivalent crosslinkers comprising said one or more sulfhydryl-reactive moieties.

21. A method of stimulating an immune response to 2-hydroxyhippuric acid, comprising:
immunizing an animal with a conjugate of claim 11.

22. A method according to claim 21, further comprising isolating one or more antibodies that specifically bind 2-hydroxyhippuric acid, wherein said binding affinity for 2-hydroxyhippuric acid is at least a factor of 10 greater than an affinity for salicylic acid.

23. A method according to claim 22, wherein said one or more antibodies are isolated directly from said animal.

24. A method of determining a 2-hydroxyhippuric acid concentration in a sample, comprising:
performing a competitive immunoassay in which a conjugate having the structure 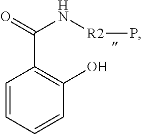
wherein R2 is a linkage chemistry and P is a detectable label, wherein R2 and P are covalently linked, and wherein the conjugate competes with 2-hydroxyhippuric acid in said sample for binding to an antibody, wherein a signal obtained from said assay is indicative of the concentration of 2-hydroxyhippuric acid in said sample; and
determining the concentration of 2-hydroxyhippuric acid in said sample from the assay signal.

25. A method of determining a 2-hydroxyhippuric acid concentration in a sample, comprising:
performing a competitive immunoassay in which 2-hydroxyhippuric acid in said sample competes for binding to an antibody raised against an immunogen having the structure 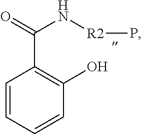
wherein R2 is a linkage chemistry and P is a carrier polypeptide, wherein R2 and P are covalently linked, wherein a signal obtained from said assay is indicative of the concentration of 2-hydroxyhippuric acid in said sample; and
determining the concentration of 2-hydroxyhippuric acid in said sample from the assay signal.

26. A method of diagnosing appendicitis in a subject, comprising,
performing a competitive immunoassay according to claim 24 on a sample from the subject; and
correlating the concentration of 2-hydroxyhippuric acid determined to the presence or absence of appendicitis in the subject.
